(19) United States
(12) Patent Application Publication
(10) Pub. No.: US 2019/0201576 A1
(43) Pub. Date: Jul. 4, 2019

(54) BIOCOMPATIBLE ALLOY AND MEDICAL PRODUCT

(71) Applicants: TOKUSEN KOGYO CO., LTD., One-city, Hyogo (JP); NATIONAL UNIVERSITY CORPORATION TOKYO MEDICAL AND DENTAL UNIVERSITY, Tokyo (JP)

(72) Inventors: Takao HANAWA, Tokyo (JP); Yusuke TSUTSUMI, Tokyo (JP)

(21) Appl. No.: 16/328,677

(22) PCT Filed: Aug. 21, 2017
(86) PCT No.: PCT/JP2017/029708

§ 371 Date: Feb. 26, 2019

(30) Foreign Application Priority Data
Sep. 8, 2016 (JP) 2016-175110

(51) Int. Cl. A61L 27/04; A61L 27/50; A61K 6/04; A61L 31/02; C22C 16/00

(52) U.S. Cl. CPC A61L 27/047; A61L 27/50; C22C 16/00; A61L 31/022; A61K 6/04

(57) ABSTRACT

Provided is a biocompatible alloy having low magnetic susceptibility and excellent mechanical properties. The biocompatible alloy according to the present invention contains: Zr as a main component; Nb of not less than 0.1% by mass and not greater than 25% by mass; Mo of not less than 0.1% by mass and not greater than 25% by mass; and Ta of not less than 0.1% by mass and not greater than 25% by mass. A total content of Nb, Mo, and Ta in the biocompatible alloy is not less than 2% by mass and not greater than 50% by mass. The biocompatible alloy has a mass susceptibility of not greater than 1.50×10⁻⁶ cm³/g. The biocompatible alloy has a Young's Modulus of not greater than 100 GPa. Various biocompatible implants and medical devices can be manufactured from the biocompatible alloy.

### TECHNICAL FIELD

The present invention relates to medical products such as biocompatible implants and medical devices, and to alloys suitable for use in these medical products.

### BACKGROUND ART

Conventionally, patients with a partial defect in their skull, cheekbone, jawbone, or the like are subjected to treatment using an artificial bone. The artificial bone is implanted in a living body to fill the defect. Patients who have lost their tooth are subjected to treatment using an artificial dental root. The artificial dental root is implanted into the jawbone. Such an artificial bone or artificial dental root is called an implant. Other than these implants, a cerebral aneurysm clip, a prosthetic heart valve, an intravascular stent, a fixing plate for fixing fractured bone fragments, etc., are implanted in a living body.

When a patient having such a biocompatible implant in their body is subjected to diagnostic imaging by a magnetic resonance imaging (MRI) diagnostic machine, errors in imaging called artifacts may appear around the biocompatible implant in the image. Such artifacts impair the precision of the diagnostic imaging.

Various medical devices are used when performing diagnostic MRI. There are cases where these medical devices cause artifacts in the image. These artifacts also impair the precision of the diagnostic imaging.

When performing diagnostic MRI, the strong magnetic field of the MRI machine causes artifacts. The artifacts can be suppressed by using materials having low magnetic susceptibility in medical products, such as biocompatible implants and medical devices. Japanese Laid-Open Patent Application Publication No. 2010-75413 discloses a biocompatible alloy that contains Zr and a main transition metal other than Zr. Specifically, this publication discloses an alloy that contains Nb of not less than 3% by mass and not greater than 12% by mass, and the rest of the alloy is Zr. Since the alloy has low magnetic susceptibility, the alloy is capable of suppressing artifacts.

### CITATION LIST

### Patent Literature

PTL 1: Japanese laid-Opera Patent Application Publication No. 2010-75413

### SUMMARY OF INVENTION

### Technical Problem

The mechanical properties of the alloy disclosed in Japanese Laid-Open Patent Application Publication No. 2010-75413 are insufficient. An object of the present invention is to provide a biocompatible alloy that has low magnetic susceptibility and excellent mechanical properties, and to provide a medical product in which the biocompatible alloy is used.

### Solution to Problem

A biocompatible alloy according to the present invention contains: Zr as a main component; Nb of not less than 0.1% by mass and not greater than 25% by mass; Mo of not less than 0.1% by mass and not greater than 25% by mass; and Ta of not less than 0.1% by mass and not greater than 25% by mass.

Preferably, a total content of Nb, Mo, and Ta in the biocompatible alloy is not less than 2% by mass and not greater than 50% by mass.

Preferably, in the biocompatible alloy, the Nb content is not less than 0.5% by mass and not greater than 25% by mass, the Mo content is not less than 0.1% by mass and not greater than 25% by mass, and the Ta content is not less than 1.0% by mass and not greater than 15% by mass.

Preferably, in the biocompatible alloy, the Nb content is not less than 12% by mass and not greater than 16% by mass, the Mo content is not less than 0.5% by mass and not greater than 5% by mass, and the Ta content is not less than 3% by mass and not greater than 12% by mass.

Preferably, a ratio (PMo/PTa) of PMo, which is the Mo content (% by mass), to PTa, which is the Ta content (% by mass), is not less than 1/20 and not greater than 1/3.

Preferably, the biocompatible alloy has a mass susceptibility of not greater than 1.50×10⁻⁶ cm³/g.

Preferably, the biocompatible alloy has a Young's Modulus of not greater than 100 GPa.

In another aspect, a medical product according to the present invention is made of the above biocompatible alloy.

### Advantageous Effects of Invention

The magnetic susceptibility of the biocompatible alloy according to the present invention is low. The biocompatible alloy makes it possible to obtain a medical product that is less likely to cause artifacts. The biocompatible alloy also makes it possible to obtain a medical product that has excellent mechanical properties.

### BRIEF DESCRIPTION OF DRAWINGS

FIG. 1 is a graph showing the results of a study, in which the compositions of Zr-based alloys were studied by using the d-electron alloy design theory.

FIG. 2 is a front view of a test piece used in a tensile test.

FIG. 3 is a front view of an ingot for Vickers hardness measurement.

FIG. 4 is a graph showing XRD results.

### DESCRIPTION OF EMBODIMENTS

Hereinafter, the present invention is described in detail based on preferred embodiments with reference to the drawings as necessar.

A medical product according to the present invention is made of a biocompatible alloy. The biocompatible alloy contains Zr, Nb, Mo, and Ta. Preferably, the rest of the alloy is made up of inevitable impurities.

The inventors of the present invention studied the compositions of Zr-based alloys by using the d-electron alloy design theory. The d-electron alloy design theory is a method in which parameters indicating the properties of alloy elements are obtained, and the parameters are used to study alloy compositions. Based on this theory, an alloy composition having intended mechanical properties can be determined.

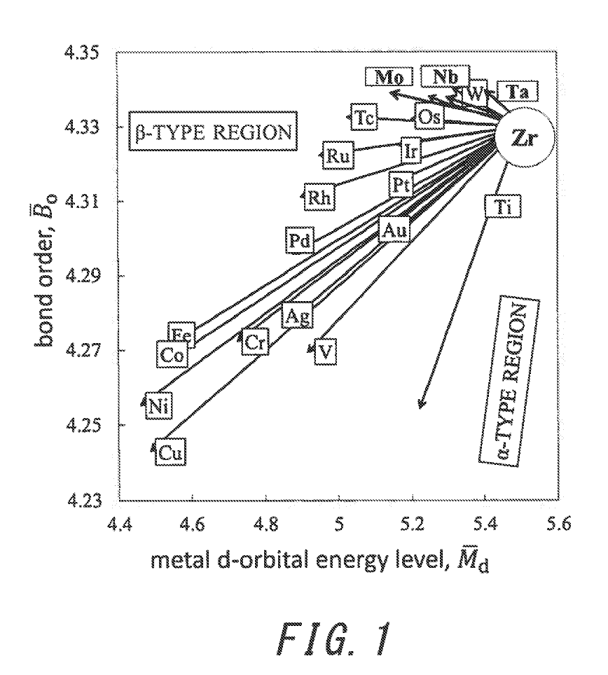

FIG. 1 is a graph showing the results of the study, in which the compositions of Zr-based alloys were studied by using the d-electron alloy design theory. In the graph, the directions of arrows contribute to phase determination, and the lengths of the arrows contribute to phase stability. It is clear from the graph that Nb, Mo, and Ta contribute to β phase formation of a Zr alloy.

Zr is a main component of the biocompatible alloy according to the present invention. Among the content rates of all the elements in the alloy, the Zr content is the highest in the alloy. Zr has low cytotoxicity. In addition, Zr has excellent corrosion resistance. Therefore, a medical product in which the alloy is used is excellent in terms of durability in a living body. In the alloy, the Zr content is preferably not less than 40% by mass, and particularly preferably not less than 50% by mass.

Nb is a β phase-stabilized element. Nb contributes to the lowering of the magnetic susceptibility of the alloy. This alloy is capable of suppressing artifacts in diagnostic MRI. Nb is capable of forming an all proportional solid solution together with Zr. Therefore, Nb can be uniformly distributed in the alloy. This alloy has excellent mechanical properties. In addition, Nb has low cytotoxicity.

The Nb content in the alloy is preferably not less than 0.1% by mass and not greater than 25% by mass. The alloy in which the Nb content is not less than 0.1% by mass is capable of suppressing artifacts. In addition, the alloy in which the Nb content is not less than 0.1% by mass is capable of contributing to the mechanical properties of a medical product. In light of these, the Nb content in the alloy is more preferably not less than 0.5% by mass, and particularly preferably not less than 12% by mass. The alloy in which the Nb content is not greater than 25% by mass is capable of suppressing artifacts. In light of this, the Nb content in the alloy is more preferably not greater than 20% by mass, and particularly preferably not greater than 16% by mass.

Mo is a β phase-stabilized element. Mo contributes to the lowering of the magnetic susceptibility of the alloy. This alloy is capable of suppressing artifacts in diagnostic MRI. Mo contributes to the mechanical properties of the alloy. In addition, Mo has low cytotoxicity.

The Mo content in the alloy is preferably not less than 0.1% by mass and not greater than 25% by mass. The alloy in which the Mo content is not less than 0.1% by mass is capable of suppressing artifacts. In addition, the alloy in which the Mo content is not less than 0.1% by mass is capable of contributing to the mechanical properties of a medical product. In light of these, the Mo content in the alloy is more preferably not less than 0.8% by mass, and particularly preferably not less than 1.0% by mass. The alloy in which the Mo content is not greater than 25% by mass is capable of suppressing artifacts. In light of this, the Mo content in the alloy is more preferably not greater than 10% by mass, and particularly preferably not greater than 5% by mass.

Ta is a β phase-stabilized element. Ta also facilitates an phase formation. Ta contributes to the lowering of the magnetic susceptibility of the alloy. This alloy is capable of suppressing artifacts in diagnostic MRI. Ta is capable of forming an all proportional solid solution together with Zr. Therefore, Ta can be uniformly distributed in the alloy. This alloy has excellent mechanical properties. In addition, Ta has low cytotoxicity.

The Ta content in the alloy is preferably not less than 0.1% by mass and not greater than 25% by mass. The alloy in which the Ta content is not less than 0.1% by mass is capable of suppressing artifacts. In addition, the alloy in which the Ta content is not less than 0.1% by mass is capable of contributing to the mechanical properties of a medical product. In light of these, the Ta content in the alloy is more preferably not less than 1.0% by mass, and particularly preferably not less than 3% by mass. The alloy in which the Ta content is not greater than 25% by mass is capable of suppressing artifacts. In light of this, the Ta content in the alloy is more preferably not greater than 15% by mass, and particularly preferably not greater than 12% by mass.

In the alloy, the total content of Nb, Mo, and Ta is preferably not less than 2% by mass and not greater than 50% by mass. The alloy in which the total content of Nb, Mo, and Ta is within this range achieves all of the following advantages: high tensile strength; high breaking elongation; low Young's Modulus; and low magnetic susceptibility. In light of this, the total content of Nb, Mo, and Ta is more preferably not less than 10% by mass, and particularly preferably not less than 15% by mass. The total content of Nb, Mo, and Ta is more preferably not greater than 40% by mass, and particularly preferably not greater than 35% by mass.

It is clear from the graph of FIG. 1 that Mo contributes to the β phase stability to a great degree, whereas Ta contributes to the β phase stability to a small degree. Therefore, preferably, in the alloy, the Ta content PTa (% by mass) is greater than the Mo content PMo (% by mass). The ratio (PMo/PTa) is preferably not less than 1/20 and not greater than 1/3, more preferably not less than 1/15 and not greater than 1/4, and particularly preferably not less than 1/10 and not greater than 1/5.

The alloy may further contain other elements in small amounts. Examples of the other elements include B, C, N, O, Na, Mg, Si, P, S, K, Ca, and Mn. The total content of the other elements in the alloy is preferably not greater than 10% by mass, more preferably not greater than 5% by mass, and particularly preferably not greater than 1.0% by mass.

The mass susceptibility of the alloy is preferably not greater than 1.50×10⁻⁶ cm³/g. The alloy having a mass susceptibility within this range is capable of suppressing artifacts. In light of this, the mass susceptibility of the alloy is more preferably not greater than 1.45×10⁻⁶ cm³/g, and particularly preferably not greater than 1.40×10⁻⁶ cm³/g. A columnar test piece is used in the measurement of the mass susceptibility. The test piece has a bottom surface diameter of 3 mm and a height of 25 mm. The measurement is performed with a manually operated magnetic balance (“MSK-MK1” available from Sherwood Scientific, Ltd.). The applied magnetic field is 0.35 T.

The Young's Modulus of the alloy is preferably not greater than 100 GPa. Assume that, for example, a fracture fixing device, an artificial joint stem, or the like is obtained from the alloy having a Young's Modulus within this range. In this case, bone resorption caused by stress shielding can be reduced. In light of this, the Young's Modulus of the alloy is more preferably not greater than 80 GPa, and particularly preferably not greater than 70 GPa. The Young's Modulus of the alloy is preferably not less than 10 GPa, and particularly preferably not less than 20 GPa, A columnar test piece is used in the measurement of the Young's Modulus. The test piece has a bottom surface diameter of 3 mm and a height of 52 mm. The measurement is performed with an elastic modulus measuring device of a free vibration type (“JE2-C1” available from Nihon Techno-Plus Corp.).

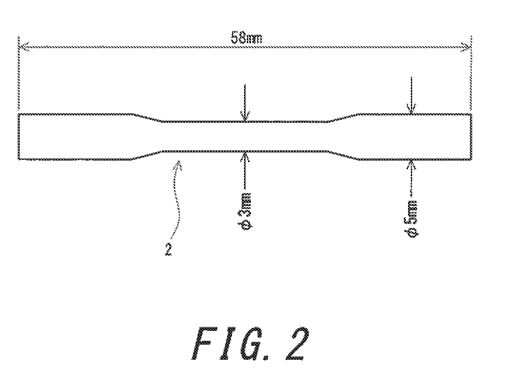

The tensile strength of the alloy is preferably not less than 600 MPa. A medical product having excellent durability can be obtained from the alloy having a tensile strength of not less than 600 MPa. In light of this, the tensile strength is more preferably not less than 700 MPa, and particularly preferably not less than 750 MPa. The tensile strength is measured by a tensile test. A test piece 2 having a shape shown in FIG. 2 is used in the tensile test. The test piece 2 is obtained by argon arc centrifugal casting. In this casting method, the internal pressure of a chamber is adjusted to be not greater than 1.2×10⁻¹ Pa, and then argon gas with a purity of not less than 99.9% is injected into the chamber to set the internal pressure to 0.06 MPa. The casting is performed in the chamber. The tensile test is performed with a precision universal testing machine (“AG-2000B” available from Shimadzu Corporation). The initial strain rate at the test is 1.3×10⁻³.

The 0.2% proof stress of the alloy is preferably not less than 550 MPa. A medical product having excellent durability can be obtained from the alloy having a 0.2% proof stress of not less than 550 MPa. In light of this, the 0.2% proof stress of the alloy is more preferably not less than 650 MPa, and particularly preferably not less than 700 MPa. The 0.2% proof stress is measured by the aforementioned tensile test.

The breaking elongation of the alloy is preferably not less than 5%. A medical product having excellent durability can be obtained from the alloy having a breaking elongation of not less than 5%. In light of this, the breaking elongation of the alloy is more preferably not less than 8%, and particularly preferably not less than 10%. The breaking elongation is measured by the aforementioned tensile test.

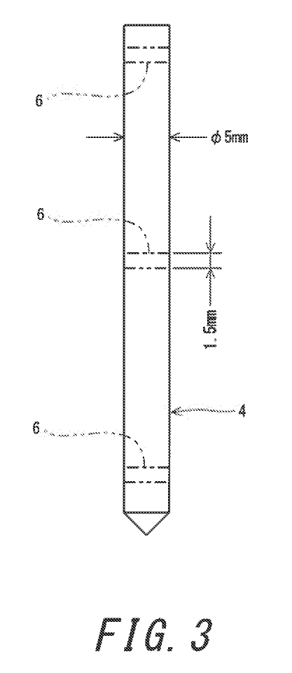

The Vickers hardness of the alloy is preferably not less than 160. A medical product having excellent durability can be obtained from the alloy having a Vickers hardness of not less than 160. In light of this, the Vickers hardness of the alloy is more preferably not less than 180, and particularly preferably not less than 200. In the measurement of the Vickers hardness, three discs 6 each having a height of 1.5 mm are cut from an ingot 4 having a shape shown in FIG. 3. The Vickers hardness is measured on the cross section of each disc 6. The measurement is performed at 12 measurement points that are randomly chosen, and the measurement results are averaged. At the time of the measurement, a load of 3 N is applied, and the dwell time of the load is 15 seconds.

The definition of the medical product according to the present invention includes biocompatible implants and medical devices. Examples of the biocompatible implants include implants such as an artificial bone, an artificial dental root, and an artificial joint. Other examples of the biocompatible implants include a fracture fixing plate, an osteosynthesis nail, an osteosynthesis screw, an intramedullary nail, a ligator (e.g., a clip), a suture instrument (e.g., a stapler), an artificial joint, a blood vessel repairing material (e.g., a stent), and a prosthetic heart valve. Even if a patient having any of these biocompatible implants in their body is subjected to diagnostic MRI, artifacts are less likely to appear in the obtained image.

Examples of the medical devices include surgical products such as a medical scalpel, medical scissors, medical tweezers, a medical spoon, a medical hook, medical forceps, a medical saw, a medical chisel, a medical raspatory, a medical hammer, a medical file, a medical pry, a medical snare, an injection needle, a puncture needle, a medical puncture instrument, a drill, a perforator, a medical tube with a beak-shaped tip (e.g., a catheter guide wire), and a body fluid induction tube. Even if any of these medical devices is used in diagnostic MRI, artifacts are less likely to appear in the obtained image.

Examples of a method of manufacturing a medical product from the alloy according to the present invention include plastic working, casting, and sintering. Examples of the plastic working include drawing and extrusion.

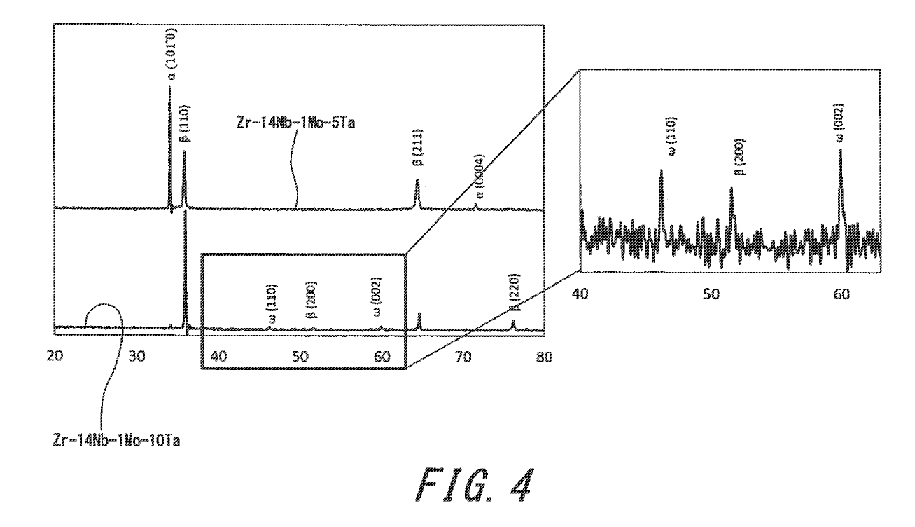

Specific examples of the alloy according to the present invention include Zr-14Nb-1Mo-5Ta and Zr-14Nb-1Mo-10Ta. FIG. 4 is a graph showing XRD results of these alloys. It is clear from this graph that α and β phases are deposited on Zr-14Nb-1Mo-5Ta, and that α, β, and ω phases are deposited on Zr-14Nb-1Mo-10Ta. It is also clear from this graph that the α phase decreases, the β phase increases, and the co phase increases in accordance with increase in the Ta content.

Tables 1 and 2 below show the properties of Zr-14Nb-1Mo-5Ta and Zr-14Nb-1Mo-10Ta together with the properties of other alloys.

TABLE 1
Property Values of Alloys

| | Zr—14Nb—1Mo—5Ta | Zr—14Nb—1Mo—10Ta | Pure Zr | Zr—14Nb |
|---|---|---|---|---|
| Vickers Hardness | 208 | 210 | 129 | 275 |
| Tensile Strength | 796 | 765 | 451 | 784 |
| (MPa) | | | | |
| 0.2% Proof Stress | 754 | 717 | 349 | 686 |
| (MPa) | | | | |
| Breaking Elongation | 15 | 11 | 13.7 | 12 |
| (%) | | | | |
| Young's Modulus | 61 | 58 | 95 | 70 |
| (GPa) | | | | |
| Magnetic Susceptibility | 1.38 | 1.35 | 1.34 | 1.35 |
| (×10⁻⁶ cm³/g) | | | | |

TABLE 2
Property Values of Alloys

| | Zr—1Mo | Ti | Ti6Al—4V ELI | Ti—6Al—7Nb | Co—Cr—Mo |
|---|---|---|---|---|---|
| Vickers Hardness | — | — | — | — | — |
| Tensile Strength | 970 | — | 980 | 933 | 980 |
| (MPa) | | | | | |
| 0.2% Proof Stress | 855 | — | 920 | 817 | 680 |
| (MPa) | | | | | |
| Breaking Elongation | 2.9 | — | 14 | 7 | 11 |
| (%) | | | | | |
| Young's Modulus | 98 | 100 | 100 | 114 | 200 |
| (GPa) | | | | | |
| Magnetic Susceptibility | 1.13 | 2.90 | 3.17 | 2.81 | 7.52 |
| (×10⁻⁶ cm³/g) | | | | | |

As shown in Tables 1 and 2, the alloys according to the present invention achieve strength, low Young's Modulus, and low magnetic susceptibility. These alloys are suitable for use in medical products.

### INDUSTRIAL APPLICABILITY

The alloy according to the present invention is suitable for use in various items that are applied to a living body when performing diagnostic MRI.

### REFERENCE SIGNS LIST

2 test piece for tensile test
4 ingot for Vickers hardness measurement
6 disc for Vickers hardness measurement

1. A biocompatible alloy comprising:
Zr as a main component;
Nb of not less than 0.1% by mass and not greater than 25% by mass;
Mo of not less than 0.1% by mass and not greater than 25% by mass; and
Ta of not less than 0.1% by mass and not greater than 25% by mass.

2. The biocompatible alloy according to claim 1, wherein
a total content of Nb, Mo, and Ta is not less than 2% by mass and not greater than 50% by mass.

3. The biocompatible alloy according to claim 1, wherein
the Nb content is not less than 0.5% by mass and not greater than 25% by mass,
the Mo content is not less than 0.1% by mass and not greater than by mass, and
the Ta content is not less than 1.0% by mass and not greater than 15% by mass.

4. The biocompatible alloy according to claim 1, wherein
the Nb content is not less than 12% by mass and not greater than 16% by mass,
the Mo content is not less than 0.5% by mass and not greater than 5% by mass, and
the Ta content is not less than 3% by mass and not greater than 12% by mass.

5. The biocompatible alloy according to claim 1, wherein
a ratio (PMo/PTa) of PMo, which is the Mo content (% by mass), to PTa, which is the Ta content (% by mass), is not less than 1/20 and not greater than 1/3.

6. The biocompatible alloy according to claim 1, wherein
the biocompatible alloy has a mass susceptibility of not greater than 1.50×10⁻⁶ cm³/g.

7. The biocompatible alloy according to claim 1, wherein
the biocompatible alloy has a Young's Modulus of not greater than 100 GPa.

8. A medical product made of the biocompatible alloy according to claim 1.
